(19) United States
(12) Patent Application Publication
(10) Pub. No.: US 2025/0375292 A1
(43) Pub. Date: Dec. 11, 2025

(54) Endovascular device with guide wire

(71) Applicant: PHENOX GMBH, Bochum (DE)

(72) Inventors: Nuran ABDULLAYEV, Köln (DE); Michael SCHÖNFELD, Köln (DE); Volker TRÖSKEN, Witten (DE); Ronald KONTEK, Bochum (DE); Hans HENKES, Stuttgart (DE); Hermann MONSTADT, Bochum (DE)

(73) Assignee: PHENOX GMBH, Bochum (DE)

(21) Appl. No.: 18/874,085

(22) PCT Filed: Jun. 12, 2023
(86) PCT No.: PCT/EP2023/065596

§ 371 Date: Dec. 11, 2024

(30) Foreign Application Priority Data
Jun. 13, 2022 (DE) 102022114767.3

(51) Int. Cl. A61F 2/24; A61F 2/915

(52) U.S. Cl. CPC A61F 2/2433; A61F 2/915

(57) ABSTRACT

The invention relates to an endovascular device with a stent structure and a guide wire. The stent structure has a substantially cylindrical main portion, and at least one point of connection with a guide wire, wherein the guide wire runs along the periphery of the stent structure and extends distally beyond the stent structure. According to an alternative embodiment, the stent structure connects to a tube through which a guide wire extends distally beyond the stent structure. The device according to the invention permits the simultaneous introduction of guide wire and stent structure into the blood vessel together with a microcatheter without there having to be, in the interim, a change between the guide wire, the microcatheter and the stent structure.

The invention relates to an endovascular device having a stent structure which is present in an expanded state, wherein it is at least temporarily released in a blood vessel, and in a compressed state, wherein it is introduced into the blood vessel through a microcatheter, wherein the stent structure has a substantially cylindrical main portion.

Thromboembolic diseases such as myocardial infarction, pulmonary embolism, peripheral thrombosis, organ embolism, etc. are typically caused by a thromboembolus (hereafter referred to as thrombus), i.e. a viscoelastic blood clot consisting of platelets, fibrinogen, coagulation factors, etc., which has become lodged in a blood vessel and completely or partially occludes it. The occlusion of organ arteries leads to an interruption in the supply of oxygen and nutrients to the dependent tissue. The disturbance of the functional metabolism with loss of function is followed within a short time by the cessation of the structural metabolism with the destruction of the affected tissue (infarction). The most common organs affected in humans are the heart and the brain. However, such changes also affect the arteries of the extremities and the pulmonary arteries. Venous thromboses and thromboembolic occlusions also occur more frequently in the leg and pelvic veins. The clinical picture of a thrombotic occlusion of an intracranial sinus can lead to severe cerebral hemorrhage due to the disruption of venous drainage of the brain tissue.

In view of the severity of the clinical pictures caused by thromboembolism and the frequency of these diseases, various techniques are known for dissolving or removing thrombi.

For example, it is known to treat patients with thrombolytic agents such as streptokinase or urokinase or with anticoagulants, which serve to thrombolize or contain thrombus growth. As these treatment methods are usually time-consuming, they are often combined with methods that serve to break up or remove the thrombus or embolus.

In addition to open surgical procedures, transluminal or endovascular catheter-guided interventional forms of therapy are increasingly being used, as they are less invasive. For example, it is known to remove the thrombus from the patient's body by means of suction catheters that generate oppression or mechanically with catheters equipped with catch baskets, coils, hooks or the like, see U.S. Pat. Nos. 6,245,089 B1; 5,171,233 A1, Thomas E. Meier et al, Stroke 2002 (9), 2232.

The disadvantage of thrombolytic treatment methods is that they are rarely successful once the time window has expired. Even the known transluminal devices are often unable to completely remove a thrombus, wherein there is also the risk that the thrombus or fragments of it are released and travel in the bloodstream to smaller lumen vessels, where they are more difficult to reach and treat.

WO 2012/156069 A1 discloses a thrombectomy device with a slot extending helically over the lateral surface of the device, wherein a clamping bracket spans the slot at the proximal end in an undulating manner. Once the thrombus has been captured, the device is withdrawn into a microcatheter (aspiration catheter) and removed from the vascular system together with the thrombus. This thrombectomy device is particularly suitable for removing thrombi from small-lumen or highly tortuous vessels such as those in the brain.

Other endovascular devices are implants which, unlike thrombectomy devices, are not intended to be removed from the blood vessel system after a thrombus has been trapped, but are intended to remain in the blood vessel permanently. Such implants are, for example, stents that are used to keep a blood vessel open, particularly after a stenosis (narrowing) in the blood vessel has been removed. The latter can be done using angioplasty (PTA) and a balloon catheter. Typically, stents have a tubular structure and are either laser-cut, resulting in a surface of struts with apertures between them, or are made of a wire mesh. Stents can be brought to the target location through a catheter and expanded there; in the case of self-expanding stents made of shape memory materials, this expansion and placement on the inner wall of the vessel takes place independently.

Another type of implant that is permanently inserted into the blood vessel is the flow diverter. These are placed in front of the neck of an aneurysm to prevent or at least significantly reduce the inflow of blood into the aneurysm, so that the aneurysm is ultimately obliterated. As a rule, flow diverters have a higher surface density than normal stents for keeping a blood vessel open. An example of a flow diverter is described in the application WO 2008/107172 A1.

Stents or flow diverters can also be used to prevent occlusive agents or emboli introduced into an aneurysm from exiting the aneurysm. Coils, i.e. small wire coils, are often used as occlusal agents. If these enter the bloodstream from an aneurysm, this can lead to considerable complications and occlusions or injuries to blood vessels located further distally. This is prevented by stents or flow diverters placed in front of the aneurysm. If a sufficient number of occlusion agents have been inserted into the aneurysm, they become entangled with one another and thus prevent each other from exiting the aneurysm, i.e. after the aneurysm has been completely filled, it may not be necessary to cover the aneurysm neck further. The technique of placing an additional endovascular device in front of the aneurysm to prevent occlusion leakage is also known as “jailing”.

Another area of application for endovascular devices is the treatment of vasospasm. A vasospasm is a spasm-like constriction of a blood vessel. This is associated with the risk that subsequent vessels are no longer supplied with sufficient blood (ischemia), which can lead to necrosis of the tissue supplied with blood by the vessels. In the cerebral region in particular, vasospasm can occur a few days after a subarachnoid hemorrhage (SAH), often as a result of the rupture of an aneurysm. A device for treating a vasospasm is known from WO 2017/207689 A1, which is substantially a stent structure which, however, does not remain permanently in the blood vessel system, but is brought to the site of the vasospasm and widened there in order to be subsequently withdrawn again. Implants that remain permanently in the blood vessel for the treatment of vasospasm are also conceivable.

As a rule, the procedure for inserting an endovascular device involves first placing a guide wire over which a microcatheter is advanced to the target position. The guide wire is then removed so that the endovascular device itself can be inserted through the microcatheter. Normally, a guiding catheter is also used, through which the microcatheter is first advanced, wherein the distal end of the guiding catheter is proximal to the actual target position, i.e. the microcatheter is advanced beyond the distal end of the guiding catheter.

In principle, this method has proven its worth, but it requires several steps and therefore a certain amount of time. Particularly for time-critical treatments such as the removal of a thrombus, it would therefore be desirable to have an endovascular device available that enables faster positioning of the device at the target location. Simplified handling would also be desirable.

The object of providing a corresponding device is achieved according to the invention by an endovascular device having a stent structure which is present in an expanded state, in which it is at least temporarily released in a blood vessel, and in a compressed state, in which it is introduced into the blood vessel in a microcatheter, wherein the stent structure has a substantially cylindrical main portion, wherein the stent structure has at least one junction with a guide wire and wherein the guide wire extends distally beyond the stent structure, the guide wire extending along the periphery of the stent structure.

The invention is based on the idea of bringing the stent structure and the guide wire to the target position together with the microcatheter in a single process. The guide wire is connected to the stent structure, wherein the guide wire extends further distally beyond the distal end of the stent structure.

The typical procedure for insertion is to insert the stent structure with the guide wire mounted in a compressed state into a microcatheter. Placement in the microcatheter is carried out in such a manner that the distal end of the guide wire protrudes distally from the microcatheter. Typically, the endovascular device is inserted into the microcatheter as far as possible. The microcatheter can then be advanced together with the endovascular device in the microcatheter in a guiding catheter and finally in the blood vessel itself in a distal direction until the stent structure has reached the target position. The release then usually takes place by retracting the microcatheter in the proximal direction while the position of the endovascular device remains unchanged or, if necessary, by further advancing the endovascular device in the distal direction while the position of the microcatheter remains unchanged. The retraction of the microcatheter and the advancement of the endovascular device can also be combined. The fact that the guide wire protrudes distally from the microcatheter means that the guide wire is still able to guide the microcatheter and the stent structure inside it through the blood vessel system. The advantage of this method is that there is no need to switch between guide wire, microcatheter and endovascular device, which is common in the prior art, making the method simpler and less time-consuming.

The guide wire runs along the periphery of the stent structure so that its function is not impaired by the guide wire itself when the stent structure expands. For example, when the endovascular device is used to remove thrombi, the stent structure can penetrate the thrombus without interfering with the guide wire that is still in place. The device can also be withdrawn with the thrombus trapped, for example into an aspiration catheter, while the stent structure remains connected to the guide wire. This also distinguishes the device according to the invention from devices known from the prior art, in some of which a device is fixed on an insertion wire, but the device is released precisely by separating the connection between the device and the insertion wire, which is not the case according to the invention.

The connection between the guide wire and the stent structure can be fixed, i.e. the guide wire remains fixed in relation to the stent structure both in terms of rotation and longitudinal displacement. Such a structure has the advantage of being particularly simple. The fixed connection between the stent structure and the guide wire can be created, for example, by gluing, welding or soldering. Since in this case it is also not possible to twist the guide wire relative to the stent structure, it is advantageous for probing, i.e. the careful advancement of the device in the distal direction through the blood vessel system, wherein the treating physician must follow bends in the respective blood vessel, if the distal portion of the guide wire is curved to the side or, even better, in the proximal direction. In other words, the distal end of the guide wire may have a J or walking stick shape. The guide wire thus follows bends in the blood vessel largely automatically, without the risk of injury to the vessel wall.

However, connections between the stent structure and the guide wire that allow degrees of freedom of movement between the guide wire and the stent structure are particularly advantageous. For example, the connection point can be a fixed bearing that allows the guide wire to rotate relative to the stent structure, but prevents longitudinal displacement. When advancing the endovascular device and the microcatheter, the treating physician can rotate the guide wire accordingly to probe the further course of a blood vessel. In this embodiment, a distal portion of the guide wire with a bend to the side or in a proximal direction is also advantageous in order to further simplify probing and prevent injury to the inner wall of the vessel.

Finally, according to a further advantageous embodiment, the connection point between the stent structure and the guide wire is a floating bearing which allows the guide wire to be displaced in the longitudinal direction and rotated relative to the stent structure. In this manner, the treating physician can not only rotate the guide wire during advancement to better follow the course of the blood vessel, but also carefully push it back and forth in relation to the stent structure and microcatheter. Typically, the longitudinal displacement of the guide wire is limited proximally and distally by stops.

According to a particularly advantageous embodiment, the stent structure has at least two connection locations with the guide wire, wherein one connection location is a fixed bearing, which allows rotation of the guide wire relative to the stent structure, and one connection location is a floating bearing, which allows displaceability of the guide wire in the longitudinal direction and rotation of the guide wire relative to the stent structure, wherein the fixed bearing and the floating bearing are spaced apart from one another in the longitudinal direction. The combination of fixed bearing and floating bearing is used to compensate for the change in length of the stent structure that occurs when the stent structure is compressed or expanded. This is the case both when the stent structure is inserted into the microcatheter and when it is released. Expansion during release of the stent structure results in shortening, whereas compression during insertion into a catheter results in stretching of the stent structure. To prevent curvature or bending of the guide wire in this context, two connection locations between the guide wire and the stent structure are advantageous, one of which remains fixed in terms of longitudinal displacement, while the other allows relative longitudinal displacement between the guide wire and the stent structure. Typically, the more distal connection location is designed as a floating bearing and the more proximal connection location as a fixed bearing; however, the reverse configuration is also possible. With regard to the floating bearing, it is again advisable to limit the longitudinal displacement, in particular by means of stops.

According to an alternative embodiment, there is provided an endovascular device having a stent structure which is present in an expanded state in which it is at least temporarily released in a blood vessel, and in a compressed state in which it is introduced into the blood vessel in a microcatheter, wherein the stent structure has a substantially cylindrical main portion and at least one connection location with a tube through which a guide wire extends which can be brought into a position in which it extends distally beyond the stent structure. According to this alternative embodiment, the stent structure is thus not connected directly to the guide wire, but to a tube through the cavity of which the guide wire runs. In this embodiment, however, the guide wire also extends distally beyond the stent structure during use in order to be able to probe the blood vessel with the aid of the guide wire and to advance the guide wire together with a microcatheter in which the endovascular device is located. The guide wire can be moved and rotated longitudinally through the tube, which further simplifies advancement through the blood vessel system and probing.

The tube to which the stent structure is connected can also be referred to as a hypotube. This is usually understood to mean a thin tube with an internal cavity or lumen, such as can be used as the proximal portion of a catheter. The tube is usually metallic and can be made of stainless steel or a cobalt-chrome alloy, for example.

To increase the flexibility of the tube, it can have slots or notches. These are typically inserted into the tube from the outside and run substantially orthogonal to the longitudinal direction. Slots or notches running diagonally to the longitudinal direction or in a spiral are also possible.

As in the first embodiment, in which the stent structure is directly connected to the guide wire, the guide wire also preferably runs along the periphery of the stent structure when the stent structure is connected to the tube, so that when the stent structure expands, its function is not impaired by the guide wire itself.

The guide wire, in turn, usefully has a distal portion that is curved to the side or in a proximal direction. This makes it easier for the treating physician to find and probe the path through the blood vessel without running the risk of injuring the blood vessel.

Irrespective of the embodiment of the invention and the configuration of the connection locations, where reference is made to a stent structure according to the invention, this is understood to mean a substantially tubular structure of the type used in a similar manner in stents. This applies regardless of whether the endovascular device is actually used as a stent in the strict sense or as a thrombectomy device, flow diverter, device for treating a vasospasm or for other purposes. The stent structure has a substantially cylindrical main portion that makes up the majority of the length of the stent structure. The stent structure can be open or closed at the proximal and distal ends. Open is understood to mean that there are no struts or wires at the respective end of the stent structure and that struts/wires are limited to the outer circumference (outer surface) of the stent structure. With a closed end, on the other hand, struts or wires are also present in the center of the stent structure. Since there are apertures between the struts or wires, even if the distal end is closed, this end is not completely sealed; blood can still flow through the apertures. At the distal end of the stent structure, it can be substantially cylindrical, as in the main portion, but a different design is also possible, for example a radially widened distal end, which can also be referred to as a trumpet shape.

The terms “proximal” and “distal” are to be understood in such a manner that when inserting the device, parts pointing towards the treating physician are referred to as proximal, while parts pointing away from the treating physician are referred to as distal. The device is thus typically advanced together with the microcatheter through the blood vessel system in the distal direction. The term “axial” refers to the longitudinal axis of the device running from proximal to distal, the term “radial” refers to planes perpendicular to this.

The stent structure, which is cylindrical at least in some areas and preferably as a whole, generally has apertures or cells distributed over the circumferential surface of the cylinder. In other words, it is a grid or mesh structure, structured from struts, bars or wires, resulting in a large number of apertures/cells on the surface of the cylinder.

A stent structure composed of interconnected bars or struts can be produced by laser cutting in a manner known in principle; in this context, one also speaks of cut structures. In this manner, a large number of apertures or a mesh structure is created within the stent structure, wherein the apertures are distributed around the circumference of the stent structure. Other manufacturing methods are also conceivable, such as electroplating or lithographic production, 3D printing or rapid prototyping.

Alternatively, the stent structure can also be a mesh structure made of wires that form a braid. The wires typically run helically along the longitudinal axis, wherein wires running in opposite directions run over and under one another at the crossing points, so that honeycomb-shaped apertures are formed between the wires. The total number of wires is preferably 8 to 128. The wires that form the mesh structure can be individual wires made of metal, but it is also possible to provide strands, i.e. several wires of small diameter that together form a filament and are preferably twisted together.

The term “aperture” or “cell” refers to the lattice structure, regardless of whether the aperture is decoupled from the environment by a diaphragm, i.e. an aperture covered by a diaphragm is also referred to as an aperture. If required, a diaphragm can be applied to the outside or inside of the grid structure. It is also possible to embed the lattice structure in a diaphragm. The diaphragms can be made of a polymer material such as polytetrafluoroethylene, polyester, polyamides, polyurethanes, polyolefins or polysulfones. Polycarbonate urethanes (PCU) are particularly preferred.

One advantage of a stent structure made of interconnected bars or struts, which is produced in particular by laser cutting, over a mesh structure made of wires is that a stent structure made of struts is less prone to length contraction during expansion than a mesh structure. In the context of use as a thrombectomy device (device for removing thrombi and clots from blood vessels), a stent structure made of interconnected struts is also advantageous in that the radial force exerted by such a stent structure is higher than that exerted by a mesh structure made of wires, with otherwise comparable structure, strut Z-wire density and strut Z-wire thickness. This is because the struts have a fixed connection at the intersections, while the wires of a mesh structure usually only run over and under one another.

The struts or wires can have a round, oval, square, rectangular or trapezoidal cross-section, wherein in the case of a square, rectangular or trapezoidal cross-section it is advantageous to round off the edges. In addition, it makes sense to electropolish the stent structure to make it smoother and more rounded and thus less traumatic. In addition, the risk of germs or other impurities adhering is reduced. It is also possible to use flat struts/wires in the form of thin strips, especially metal strips.

The diameter of the stent structure in the freely expanded state is typically in the region of 2 to 8 mm, preferably in the region of 4 to 6 mm. The total length of the stent structure in the expanded state is generally 5 to 50 mm, preferably 10 to 45 mm, more preferably 20 to 40 mm.

In the case of a stent structure made of struts, for example, this can be cut from a tube with a wall thickness of 25 to 70 μm; in the case of a mesh structure made of interwoven wires, the wire thickness is preferably 20 to 70 μm. A microcatheter with which the device can be brought to the target location in a compressed state has an inner diameter of 0.4 to 0.9 mm, for example.

Advantageously, the stent structure has a tapered proximal portion proximal to the main cylindrical portion, which tapers towards a connection location with the guide wire or tube located on the periphery of the stent structure. Accordingly, the guide wire can run straight in the longitudinal direction of the device, namely along the periphery of the stent structure. In this context, the periphery of the stent structure refers to the circumferential surface of the stent structure, which can also be referred to as the lateral surface. The eccentric placement of the guide wire is also advantageous in that it prevents the guide wire from interfering with the functions of the endovascular device and the stent structure or obstructing the blood flow. The eccentric arrangement of the guide wire also makes it easier to withdraw the device into the microcatheter, if this proves necessary, or into an aspiration catheter.

It is advantageous if the distal portion of the guide wire has as uniform a curvature as possible to the side or in the proximal direction. This makes it easier to follow even narrow, highly curved blood vessels with the guide wire. The distal end of the guide wire is therefore not simply pointed in the distal direction, but has a rounded shape, for example a J-shape. The curvature of the distal portion of the guide wire is of particular importance if there are few or no degrees of freedom with regard to the movement between the guide wire and the stent structure, i.e. in particular if there is a fixed connection between the guide wire and the stent structure.

In addition, it is advantageous if the guide wire has a certain flexibility, particularly in its distal portion. This can be ensured by an appropriate selection of materials. For example, a shape memory metal with superelastic properties, such as a nickel-titanium alloy, can be used as the material for the guide wire. One such alloy is known as Nitinol. Other preferred materials for the guide wire are stainless steel or cobalt-chrome alloys. In particular, a distal portion of the guide wire can also be made of a particularly flexible material such as a shape memory metal, while more proximal regions can be made of a different material such as stainless steel.

Furthermore, it is advantageous to make at least portions of the distal portion of the guide wire X-ray visible. This helps the treating physician to follow the advancement of the guide wire and thus also the stent structure and the microcatheter in the X-ray image. For example, parts of the guide wire can have a platinum or platinum-iridium marking or even a gold coating.

It is expedient for the stent structure to be self-expandable in order to automatically assume the expanded state after release from the microcatheter. To ensure this, the stent structure can be structured in particular from a material with shape memory properties or at least contain such properties. Nickel-titanium alloys such as Nitinol are also suitable here. However, polymers with shape memory properties or other alloys, such as nickel-titanium-chromium or nickel-titanium-copper alloys, are also conceivable. The use of cobalt-chromium or cobalt-chromium-nickel alloys is also possible.

The cylindrical main portion is typically formed from a plurality of cells distributed around its circumference, which are open in a radial direction. In other words, the main cylindrical portion, but often the entire stent structure, has a grid or mesh structure structured of struts or wires, wherein cells are located between the struts or wires.

The resulting cells in the stent structure can be closed all around, i.e. surrounded by struts or wires without interruptions (so-called “closed-cell design”). An “open-cell design” is also possible, in which at least some struts/wires have an interruption so that the cells formed by the struts/wires are at least partially open, i.e. not completely closed. Such an open-cell design has a higher flexibility, which can be advantageous for highly tortuous blood vessels. On the other hand, the retractability of the device is limited in an open-cell design.

It is also possible to provide the stent structure or the cylindrical main portion with a slot which extends helically over the lateral surface of at least parts of the stent structure or in the longitudinal direction, i.e. substantially parallel to the longitudinal axis, along the lateral surface of at least parts of the stent structure. Individual struts or wires can span the slot in order to influence the radial force curve. A corresponding thrombectomy device with a continuous slot parallel to the longitudinal axis is disclosed in WO 2009/105710 A1, and a device with a helical slot and a clamping bracket at the proximal end of the stent structure is disclosed in WO 2012/156069 A1.

To make it easier for the treating physician to visualize the insertion process, it makes sense to provide at least some regions of the endovascular device with X-ray visible markings. The X-ray visible markings can be made of platinum, palladium, platinum-iridium, tantalum, gold, tungsten or other X-ray visible metals. For example, X-ray visible/X-ray tight coils can be attached to different points of the device. It is also possible to provide the stent structure, in particular the struts or wires of the stent structure, with a coating of a X-ray visible material, for example with a gold coating. This can have a thickness of 1 to 6 μm, for example. The coating with an X-ray visible material does not have to cover the entire stent structure; it is particularly important in the regions of the stent structure that expand towards the inner wall of the vessel, i.e. substantially in the main cylindrical section of the stent structure, in order to be able to observe the expansion of the stent structure. However, even if an X-ray visible coating is provided, it may be useful to additionally apply one or more X-ray visible markings to the device, in particular at the distal end of the stent structure.

An additional option is to use struts or wires made of a metal with shape memory properties, in particular a corresponding nickel-titanium alloy, which in any case partially have a platinum core. Such struts/wires are known as DFT wires (DFT=drawn filled tubing). In this manner, the advantageous properties of nickel-titanium on the one hand, namely the shape memory properties, are combined with the advantageous properties of platinum on the other hand, namely the X-ray visibility.

Loose strut or wire ends may be present at the proximal and distal ends of the stent structure, although these should preferably be atraumatic in order to avoid injury to the blood vessel. The atraumatic design of the ends can be achieved, for example, by rounding them off. Another option is to have the struts or wires form a loop at one or both ends of the stent structure and return them to the stent structure. Accordingly, the end of the stent structure no longer has free strut or wire ends, which reduces the risk of injury to the blood vessel wall.

In particular, the endovascular device according to the invention may be a device for removing thrombi from blood vessels. Such a device is placed inside the microcatheter at the site of the thrombus to be removed and released from the microcatheter, whereupon the stent structure of the device expands into the thrombus and absorbs the thrombus. The endovascular device is then withdrawn with the captured thrombus, typically into an aspiration catheter that has a larger inner diameter than the microcatheter to prevent the thrombus or parts of the thrombus from being “squeezed out” of the stent structure when it is compressed. At the same time, any detached thrombus fragments can be aspirated.

The invention is particularly suitable for a thrombectomy device because the removal of a thrombus that may cause an ischemic stroke is time-critical. By dispensing with several successive steps with alternation between guide wire, microcatheter and thrombectomy device, valuable time can be saved according to the invention, namely by inserting the microcatheter and the thrombectomy device into the blood vessel at the same time as the guide wire. Recanalization is therefore possible more quickly. As the guide wire runs along the periphery of the stent structure, it has virtually no influence on the effectiveness of the thrombectomy device on site. Once the thrombus has been captured, the entire thrombectomy device, including the guide wire, is removed from the blood vessel system; it is therefore not usually necessary to separate the guide wire from the thrombectomy device.

Nevertheless, the endovascular device according to the invention can also be an implant, in particular a stent for keeping blood vessels open, a flow diverter for inhibiting the inflow of blood into an aneurysm or a stent which is intended to prevent occlusion agents from escaping from the aneurysm. Another possible use is as a device for treating a vasospasm, wherein the treatment can be based in particular on a temporary or permanent expansion of the stent structure (cf. e.g. WO 2017/207689 A1) or on the application of electrical, high-frequency or ultrasonic pulses (cf. WO 2018/046592 A1).

In the case of an implant in particular, it must be possible to separate the stent structure so that it can remain in the blood vessel. It is preferable to provide a separability of the stent structure from the guide wire or tube so that the guide wire/tube can be withdrawn from the blood vessel system while the stent structure remains at the target position. In this manner, effects of the guide wire that influence the function of the stent structure are excluded. Accordingly, the connection locations between the guide wire/tube and the stent structure should be separable, wherein various separation mechanisms are known from the state of the art, in particular electrolytic, mechanical, thermal and chemical separation options. Detachability may also be useful for endovascular devices that are actually intended to be removed from the vasculature, in particular thrombectomy devices and devices for treating vasospasm, in the event that retraction of the device proves problematic during treatment and the physician decides to leave the device in the blood vessel.

The detachment location(s) for separating the stent structure are preferably electrolytically corrodible detachment locations. In this case, at least partial dissolution of the detachment location is achieved by applying an electrical voltage to the detachment location using a voltage source. In electrolytic detachment, the detachment location is electrolytically corroded by applying a voltage so that the stent structure detaches from the guide wire. This is usually direct current, wherein a low current (<3 mA) is sufficient. The detachment location is usually made of metal and forms the anode when the electrical voltage is applied, at which the oxidation and thus the dissolution of the metal takes place.

To avoid anodic oxidation of the stent structure, it can be electrically insulated from the detachment location and the guide wire. The electrolytic detachment of implants is well known from the prior art, for example for occlusion coils for closing aneurysms, cf. B. WO 2011/147567 A1. The principle is based on the fact that when a voltage is applied, a detachment location made of a suitable material, in particular metal, usually undergoes at least such extensive dissolution by anodic oxidation that the regions of the device located distal to the corresponding detachment location are released. The detachment location can be made of stainless steel, magnesium, magnesium alloys or a cobalt-chromium alloy, for example. A particularly preferred magnesium alloy is Resoloy®, which was developed by the company MeKo from Sarstedt/Germany (cf. WO 2013/024125 A1). It is an alloy of magnesium and lanthanides, in particular dysprosium. Another advantage of using magnesium and magnesium alloys is that the retention of magnesium residues in the body is physiologically unproblematic.

While the detachment location serves as the anode, the cathode can be positioned on the body surface, for example. Alternatively, another region of the device can also form the cathode. Of course, the detachment location must be connected to the voltage source in an electrically conductive manner. The guide wire itself can serve as a conductor. As the resulting corrosion current is controlled by the surface of the cathode when the cathode is placed on the surface of the body, the surface of the cathode should be significantly larger than the surface of the anode. To a certain extent, the speed of the detachment location can be controlled by adjustment of the cathode surface in relation to the anode surface. The device according to the invention can thus also comprise a voltage source and, if necessary, an electrode that can be placed on the body surface.

As an alternative to a detachment location to be dissolved electrolytically, other detachment locations known from the prior art can also be used, in particular mechanically, thermally or chemically separable detachment locations. In the case of mechanical detachment, there is typically a form, force or frictional connection that is released when the stent structure is released, causing the stent structure to detach from the guide wire or tube. In the case of a thermal detachment location, the connection can be broken by heating the detachment location, whereupon it softens or melts to such an extent that separation occurs. Finally, chemical detachment is also possible, in which the detachment is brought about by a chemical reaction at the detachment location.

The different types of detachment, for example electrolytic and mechanical detachment, can also be combined with one another. A mechanical connection is established between the units, in particular via a positive fit, which remains in place until an element maintaining the mechanical connection is electrolytically corroded.

At least parts of the stent structure can have a coating that prevents the accumulation of thrombocytes. Such an antithrombogenic coating may have a functional layer, wherein the functional layer comprises at least one sugar alcohol and/or is formed by an oligo- or polymerization of monosaccharides functionalized with polymerizable groups. Such an antithrombogenic coating is disclosed in WO 2018/210989 A1.

The device according to the invention can be used in particular in the neurovascular region, but it is also possible to use it in the cardiovascular or peripheral region.

In addition to the endovascular device, the invention also relates to a method for introducing the endovascular device according to the invention into a blood vessel, wherein the device is introduced into a microcatheter and placed in such a manner that the distal end of the guide wire protrudes distally from the microcatheter, and the microcatheter is advanced distally in the blood vessel together with the endovascular device located in the microcatheter. Before the endovascular device with the microcatheter is advanced to the target position, a relatively large-lumen guiding catheter is often used first, through which the small-lumen microcatheter is then advanced further in the distal direction. In neurovascular applications, for example, the guiding catheter can be used to advance from the groin to the carotid artery; further advancement is then carried out using the microcatheter only.

Furthermore, the invention also relates to a combination of an endovascular device and a microcatheter, wherein the endovascular device is placed in the microcatheter in such a manner that the distal end of the guide wire protrudes distally from the microcatheter.

Finally, the invention also relates to the use of the device according to the invention for removing a thrombus from a blood vessel or a corresponding method.

All descriptions of features of the invention relate in each case to all embodiments, in particular both to the first embodiment, in which the stent structure is connected to the guide wire via the connection location, and to the alternative embodiment, in which there is a connection location to a tube, unless otherwise apparent from the context.

The invention is explained in more detail with reference to the examples of embodiments illustrated in the figures. It should be noted that the figures show preferred embodiment variants of the invention, but the invention is not limited thereto. In general, the invention includes any combination of the technical features listed in the claims or described in the description as being relevant to the invention, as far as technically useful.

In the figures:

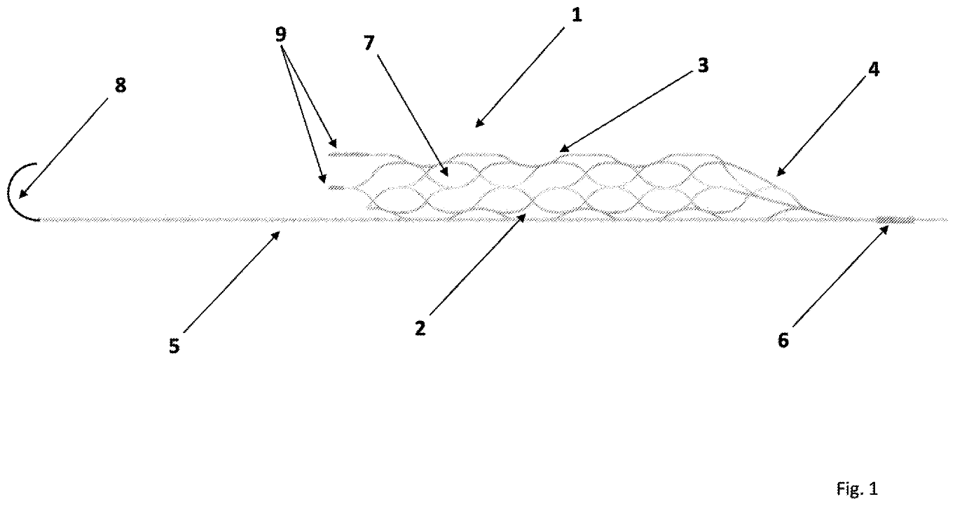

FIG. 1 shows a side view of the device according to a first embodiment of the invention;

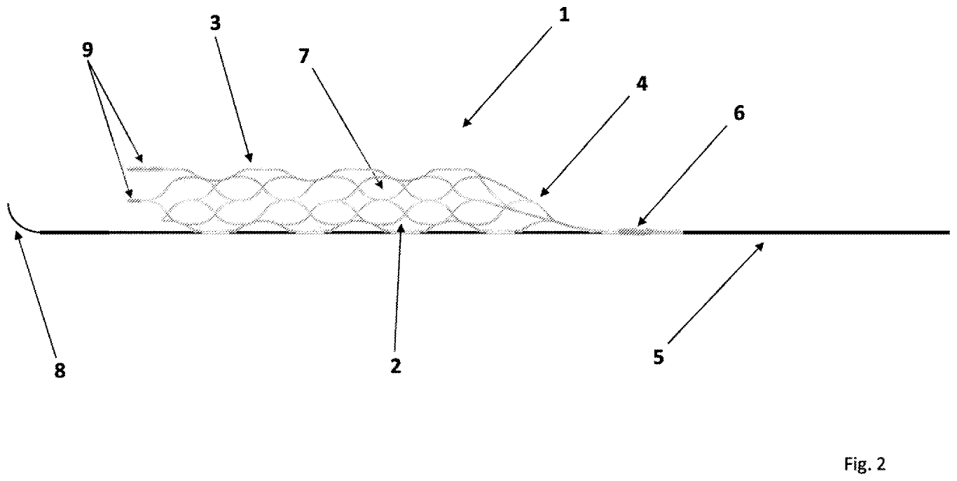

FIG. 2 shows a side view of the device according to a second embodiment of the invention;

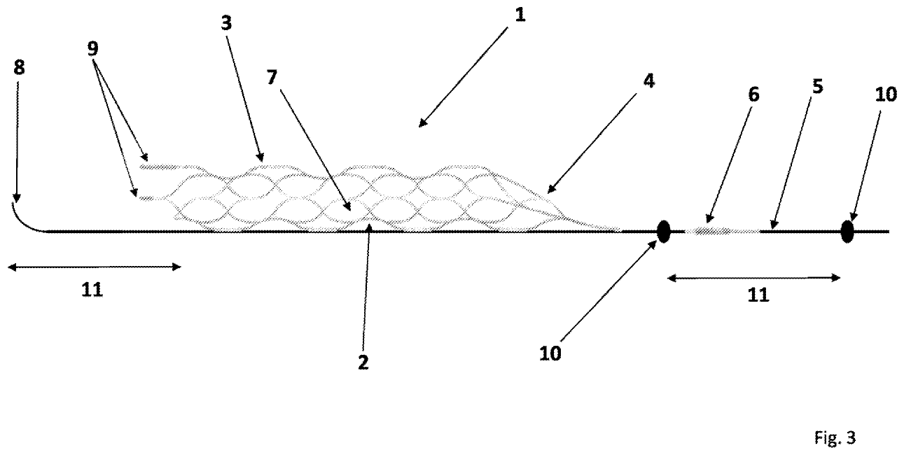

FIG. 3 shows a side view of the device according to a third embodiment of the invention; and

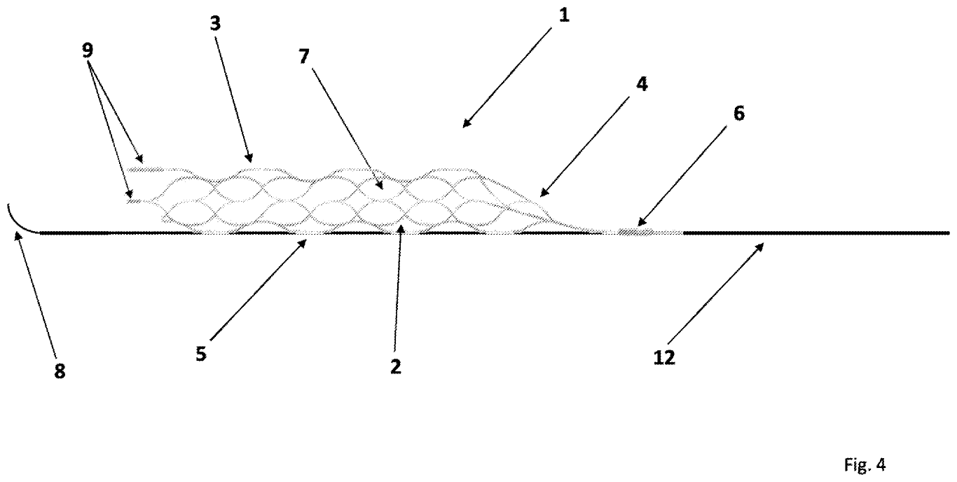

FIG. 4 shows a side view of the device according to an alternative embodiment of the invention.

FIG. 1 shows a side view of a first embodiment of the endovascular device 1 according to the invention. The endovascular device 1 is used to trap a thrombus and consists substantially of a stent structure 2 and a guide wire 5. The stent structure 2 is shown here in the expanded state, the guide wire 5 runs along the periphery on the side of the stent structure 2. Guide wire 5 and stent structure 2 are connected to each other via a connection location 6, wherein in this embodiment the connection location 6 is a fixed connection.

The stent structure 2 has a cylindrical main portion 3 and a proximal portion 4 tapering towards the periphery of the stent structure 2, where the struts of the stent structure 2 terminate in the connection location 6 with the guide wire 5. The struts of the stent structure 2 form a plurality of cells 7 distributed over the circumferential surface of the stent structure, which are open in the radial direction so that they can penetrate into a thrombus during expansion. The guide wire 5 protrudes distally well beyond the stent structure 2 to fulfill its function of guiding the device 1 through the vascular system. The distal portion 8 of the guide wire 5 is J-shaped to further facilitate probing and minimize the risk of injury to blood vessels. To visualize the insertion and release process, the stent structure 2 is provided with X-ray tight markings 9 at its distal end.

FIG. 2 shows a second embodiment of the invention. In contrast to the first embodiment, the connection location 6 is designed here as a fixed bearing, which allows the guide wire 5 to rotate relative to the stent structure 2. This further simplifies the introduction of the endovascular device 1 into the blood vessel system. Here too, the guide wire 5 protrudes significantly beyond the stent structure 2 in the distal direction and has a distal portion 8 with a curvature to the side.

Finally, FIG. 3 shows a third embodiment of the invention. Here, the connection location 6 is designed as a floating bearing, which not only allows the guide wire 5 to rotate relative to the stent structure 2, but also a limited displacement in the longitudinal direction. The longitudinal displacement is symbolized by the arrows 11 and limited by the two stops 10. The additional degree of freedom gives the treating physician additional options when inserting and advancing the endovascular device 1 in the blood vessel system.

FIG. 4 shows the alternative embodiment of the invention, wherein the stent structure 2 is not connected to the guide wire 5 via the connection location 6, but to a tube 12, wherein the guide wire 5 extends through the interior of the tube 12 and extends distally beyond the stent structure 2. The guide wire 5 is rotatable within the tube 12 and can be moved in the longitudinal direction. In this embodiment, too, the endovascular device 1 is placed in a microcatheter not shown here in such a way that the guide wire 5 projects out of the microcatheter in the distal direction so that the guide wire 5 finds its way through the blood vessel and enables probing.

1. An endovascular device having a stent structure (2) which is present in an expanded state in which it is at least temporarily released in a blood vessel and in a compressed state in which it is introduced into the blood vessel in a microcatheter, wherein the stent structure (2) has a substantially cylindrical main portion (3),
characterized in that,
the stent structure (2) has at least one connection location (6) with a guide wire (5) running along the periphery of the stent structure (2) and extends distally beyond the stent structure (2).

2. The endovascular device according to claim 1, characterized in that the at least one connection location (6) between the stent structure (2) and the guide wire (5) is a fixed bearing which allows rotation of the guide wire (5) relative to the stent structure (2).

3. The endovascular device according to claim 1, characterized in that the at least one connection location (6) between the stent structure (2) and the guide wire (5) is a floating bearing which allows the guide wire (5) to be displaced in the longitudinal direction and the guide wire (5) to be rotated relative to the stent structure (2).

4. The endovascular device according to claim 3, characterized in that the displaceability of the guide wire (5) in the longitudinal direction is limited proximally and distally by stops (10).

5. The endovascular device according to claim 1, characterized in that the stent structure (2) has at least two connection locations (6) with the guide wire (5), wherein a first connection location (6) is a fixed bearing which allows rotation of the guide wire (5) relative to the stent structure (2), and a second connection location (6) is a floating bearing which allows the guide wire (5) to be displaced in the longitudinal direction and the guide wire (5) to be rotated relative to the stent structure (2), wherein the fixed bearing and the floating bearing are spaced apart from one another in the longitudinal direction.

6. An endovascular device having a stent structure (2) which is present in an expanded state in which it is at least temporarily released in a blood vessel and in a compressed state in which it is introduced into the blood vessel in a microcatheter, wherein the stent structure (2) has a substantially cylindrical main portion (3),
characterized in that,
the stent structure (2) has at least one connection location (6) with a tube (12) through which runs a guide wire (5) along a periphery of the stent structure (2), which can be brought into a position in which the guide wire extends distally beyond the stent structure (2).

7. (canceled)

8. The endovascular device according to claim 6, characterized in that the tube (12) is made of a metal.

9. The endovascular device according to claim 6, characterized in that the tube (12) has slots or notches extending substantially orthogonally or diagonally to the longitudinal direction, which increase the flexibility of the tube (12).

10. The endovascular device according to claim 1, characterized in that the stent structure (2) has a tapering proximal portion (4) proximal to the main cylindrical portion (3), which tapers towards the at least one connection location (6) with the guide wire (5).

11. The endovascular device according to claim 1, characterized in that the guide wire (5) has a distal portion (8) which is curved to the side or in the proximal direction.

12. The endovascular device according to claim 1, characterized in that at least portions of the distal portion (8) of the guide wire (5) are designed to be X-ray visible.

13. The endovascular device according to claim 1, characterized in that the stent structure (2) is designed to be self-expandable and assumes the expanded state independently after release from a microcatheter.

14. The endovascular device according to claim 1, characterized in that the endovascular device (1) is a device for removing thrombi from blood vessels.

15. (canceled)

16. The endovascular device according to claim 1, characterized in that the at least one connection location (6) between the stent structure (2) and the guide wire (5) is designed to be electrolytically, mechanically, thermally or chemically separable.

17. A combination of an endovascular device (1) according to claim 1 and a microcatheter, wherein the endovascular device (1) is placed in the microcatheter in such a way that the distal end of the guide wire (5) protrudes distally from the microcatheter.

18. A method for introducing the endovascular device (1) according to claim 1 into a blood vessel, characterized in that the endovascular device (1) is introduced into a microcatheter and placed in such a manner that the distal end of the guide wire (5) projects distally from the microcatheter, and the microcatheter together with the endovascular device (1) located in the microcatheter is advanced in the blood vessel in the distal direction.

19. The endovascular device according to claim 6, characterized in that the displaceability of the guide wire (5) in the longitudinal direction proximally and distally is limited by stops (10).

20. The endovascular device according to claim 3, characterized in that the floating bearing is a tube joining to the stent structure through which a guide wire (5) runs.
